(19) United States
(12) Patent Application Publication
(10) Pub. No.: US 2010/0069720 A1
(43) Pub. Date: Mar. 18, 2010

(54) SPECTROSCOPICALLY ENHANCED IMAGING

(75) Inventors: Stephen Fulghum, Marblehead, MA (US); Charles Von Rosenberg, Belmont, MA (US); Michael Feld, Jamaica Plain, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: NEWTON LABORATORIES, INC., Belmont, MA (US)

(21) Appl. No.: 12/516,940

(22) PCT Filed: Nov. 30, 2007
(86) PCT No.: PCT/US07/24653

§ 371 Date: May 29, 2009

Related U.S. Application Data
(60) Provisional application No. 60/861,871, filed on Nov. 30, 2006.
(60) Provisional application No. 60/874,650, filed on Dec. 13, 2006.

(51) Int. Cl. A61B 1/06

(52) U.S. Cl. 600/175; 600/182

(57) ABSTRACT

The present invention provides systems and methods for the spectroscopic determination of the physical characteristics of the tissue under observation by an autofluorescence or other endoscope without the requirement of contacting the tissue directly. The optical probe contained in the endoscope itself is passive and may be either built into the endoscope or positioned in a biopsy channel of same. The spectroscopic information, combined with other information provided by the endoscope such as total fluorescence, improves the sensitivity and specificity of the identification of precancerous or cancerous lesions.

### CROSS REFERENCE TO RELATED APPLICATIONS

This application claims the priority of U.S. Provisional Application No. 60/861,871 filed on Nov. 30, 2006 and entitled SPECTROSCOPICALLY ENHANCED IMAGING; and U.S. Provisional Application No. 60/874,650 filed on Dec. 13, 2006 and entitled SPECTROSCOPICALLY ENHANCED IMAGING, which are hereby incorporated by reference herein.

### STATEMENT REGARDING FEDERALLY SPONSORED RESEARCH OR DEVELOPMENT

N/A

### BACKGROUND OF THE INVENTION

Autofluorescence imaging endoscopes can detect precancerous and cancerous lesions in the lung, colon and other body areas. Normal tissue, when illuminated with ultraviolet or violet light, will emit relatively weak fluorescence in the visible spectrum. This autofluorescence can be imaged by endoscopes which are not sensitive to, or which filter out, the much stronger excitation light. Precancerous and cancerous tissue, for a number of reasons such as increased hemoglobin concentration, exhibit reduced fluorescence when so visualized. Visual detection of this reduced fluorescence can identify such tissue with a high sensitivity which is useful for directing biopsies for later examination by pathologists.

High sensitivity is necessary for optimal screening of likely cancer sites. A high sensitivity means that the screening method will almost always identify a cancerous or precancerous tissue site even though it may sometimes identify normal tissue as cancerous. Fewer unnecessary biopsies would be taken, however, if the method also had high specificity, meaning that it would rarely identify normal tissue as cancerous.

### SUMMARY OF THE INVENTION

The present invention describes a passive optical system, comprising of optical fibers and lenses, which can either be built into a autofluorescence endoscope or inserted into an existing endoscope by inserting it within an existing endoscope channel. The active components of the system, including light sources, optical filters and detectors, are contained in a separate housing or within the endoscope light source enclosure. This system provides for both improved specificity and sensitivity in the spectroscopic measurement of tissue with an endoscope system.

The optical components include one or more optical fibers for collecting light emitted or reflected from the tissue and delivering it to a remote detection system. There are also one or more optical fibers for delivering remotely-generated light to the tissue either as part of a diagnostic method or simply as a visual marker for the area of tissue being optically sampled. The polished ends of both sets of fibers are preferably held in the same optical plane and are imaged together onto the tissue with a lens assembly held in a fixed position and orientation relative to the distal end of the endoscope preferably flush with the distal tip of the endoscope. If the distal tip of the probe is at or near the correct focal distance from the tissue, the images of the delivery and collection fibers do not overlap and the delivered light can not be reflected directly back into a collection fiber. If the distal tip of the probe is not close to the focal distance from the tissue the out-of-focus images of the delivery and collection fibers may overlap. This overlap may either be useful or deleterious depending upon the spectroscopic method being employed. Note that in either case the fiber-lens combination does not directly contact the tissue and thus cannot alter or damage tissue in the way that contact probes are prone to do.

The optical axis of this fiber-lens assembly is nominally parallel with the optical axis of the endoscope. It is offset laterally and fixed in this relative position so that the apparent position of the fiber images on the tissue can be correlated to the distance of the distal tip to the tissue for a specific endoscope lens/detector combination. The distal end of the probe can be inserted into a biopsy channel at the beginning of a procedure but are then held in a fixed position during the procedure. Positioning the collection area for the non-contact spectroscopic probe is thus accomplished by moving the distal tip of the endoscope until the projected marker laser spots are in the correct position on the tissue and simultaneously at the calibrated position on the video monitor of the endoscope. This is a sufficient condition to have the non-contact probe correctly focused onto the tissue.

The optical system described may be coupled to a number of different light sources and detection systems depending on the specific tissue being analyzed and the analysis method being used. This design allows a single optical system to be designed into the endoscope and optionally used with all of the following analysis and detection systems which may be switched depending on the tissue type being surveyed.

The simplest detection system can be a single optical detector such as a photodiode, avalanche photodiode or photomultiplier coupled to all of the light collection fibers. This system is appropriate, for instance, in quantifying the absolute fluorescence power from the tissue excited by the autofluorescence endoscope's own ultraviolet or violet light source. In this case the detector, like the endoscope itself, can use an optical filter to block the much stronger excitation light. Absolute total fluorescence is a diagnostic for the presence of precancers and cancer.

In this case, a visible diode laser which is not blocked by any filters in the endoscope optics, can be coupled into the delivery fibers and thus imaged onto the tissue to mark that area of the tissue from which light is being collected by the collection fibers. The position of the collection area on the tissue is set by the position of the distal end of the endoscope.

In another embodiment, an imaging spectrometer with a two-dimensional array detector, such as a CCD or CMOS imaging detector, can be used to measure the spectrum returned by each collection fiber separately. This system can be used for measuring the induced fluorescence spectrum and the white light reflected spectrum (color) of the tissue. An estimate of the local hemoglobin concentration can be obtained from the white light spectrum and used to estimate what the fluorescence signal is in the absence of that hemoglobin. A fluorescence spectrum is a superior cancer diagnostic to the total fluorescence power alone. An estimate of the hemoglobin concentration of the tissue is also a diagnostic of cancer and precancer.

The delivery fibers can be used to simply indicate the area of the tissue that is being analyzed. Alternatively, the delivery fibers can be used to couple narrow-band laser light into the tissue at those points on the tissue where the distal tips of the delivery fibers are imaged. The collection fibers are imaged at different spots on the tissue, separate from those areas where the narrow-band laser light enters the tissue. The scattering through the tissue can thus be measured. The local hemoglobin concentration can be measured by comparing the scattering in the tissue at several wavelengths, specifically where hemoglobin absorption is significant and at wavelengths where it is not significant. Imaging spectrometers can separate the light exiting one collection fiber from another and have sufficient dispersion to separate laser sources from each other. In the preferred embodiment of this detection system three delivery fibers, three collection fibers and six laser wavelengths are used to obtain 18 different combinations of wavelength and scattering distance in a single exposure. This allows a much more precise measurement of both the scattering spectrum in the tissue and the hemoglobin concentration in the tissue. Superior measurements will yield more precise predictions of the likely presence or absence of cancer.

Imaging spectrometers and thermo-electrically-cooled, two-dimensional CCD's are sensitive but relatively slow because of the time required to digitize the signal in each pixel. Faster CMOS imaging arrays are available but can have higher noise levels. When a high resolution spectrum is not required or when the illumination source is a laser, the detectors can be made with optical filters and high speed photomultipliers. These detection systems can return quantified results in less than a second which may be important if measurements need to be taken quickly in succession, such as for comparing measurements in one tissue area to measurements in a neighboring area. A preferred embodiment of this type of detection system utilizes three delivery fibers, a plurality of light sources such as, six laser light sources, three collection fibers and a rotating three-color filter wheel. The same 18 combinations of scattering distances and colors described in imaging spectrometer system above can be obtained in a smaller, less expensive package and with a reduced collection time.

### BRIEF DESCRIPTION OF THE DRAWINGS

Preferred embodiments of the present invention are described with reference to the following drawings, wherein:

FIG. 1 is a side-view schematic diagram of the passive optical components of a system as contained in the distal tip of an endoscope showing the relative foci of the lens systems.

FIG. 2A is an end-view schematic diagram of the passive optical components of the system as contained in the distal tip of an endoscope.

FIG. 2B is a preferred arrangement of the delivery and collection fibers.

FIG. 2C is an optical ray trace showing the imaging of the system delivery fibers onto the tissue and the imaging of the tissue area being measured onto the endoscope imaging detector.

FIG. 2D shows an optical ray trace of the distribution fibers and the collection fibers as they are imaged onto the tissue.

FIG. 3A shows a preferred arrangement of the delivery fibers and collection fibers including one option for their relative sizes.

FIG. 3B shows a detection method wherein light from the collection fibers is measured with a single detector and light delivered to the tissue is generated by a single illumination source.

FIG. 3C shows a detection method wherein light from the collection fibers is dispersed and imaged onto a 2-dimensional array detector by an imaging spectrometer and a method by which two or more light sources can be coupled into a single delivery fiber.

FIG. 3D shows a detection method wherein light from the collection fibers are measured by single detectors for each collection fiber with a rotating filter wheel interspersed between them.

FIG. 4A shows an optical ray trace indicating that light from the delivery fibers is imaged onto the tissue then scattered and reimaged onto the endoscope detector.

FIG. 4B shows the illuminated spots on the tissue as seen by the endoscope image display device.

FIG. 4C shows how the illuminated spots on the tissue move on the endoscope image display device as a function of the distance of the distal tip from the tissue.

FIG. 5A shows an optical ray trace of a simulated endoscope tissue subject as illuminated by the system's delivery fibers.

FIG. 5B shows the image of the simulated tissue subject as seen, when inverted, on the endoscope image display device.

FIG. 6A shows an optical ray trace diagram of scattered light exiting the tissue after being illuminated by a single delivery fiber.

FIG. 6B shows the relative scattering distances of light which enters the tissue at a delivery spot and exits the tissue at each of the three collection spots.

FIG. 6C shows a graph of both measured and simulated signals from the collection fibers as a function of tissue scattering coefficient, including a projected signal variation for tissue with an intrinsic absorption.

FIG. 7A shows typical laser line sources overlaid on the absorption spectrum of hemoglobin which is the dominant absorber in tissue.

FIG. 7B shows how laser light from multiple line sources delivered to the tissue and scattered through the tissue can be collected by multiple collection fibers, dispersed by an imaging spectrometer and measured with a 2-dimensional array detector in a single exposure.

FIG. 7C shows how the same number of laser lines, delivery fibers and collection fibers require a rotating filter wheel and separately timed exposures if discrete photodetectors are used.

FIG. 8 shows the multiple tissue scattering paths and colors are collected simultaneously by the imaging spectrometer detection method.

FIG. 9 shows how the same number of tissue scattering paths and colors require six sequential exposure periods when the rotating filters and discrete photodetector detection method is used.

FIGS. 10A-C shows the results of an optical model of tissue scattering indicating that the ratio of the measured scattering signals, as plotted in FIG. 6, will vary slightly with the distance of the probe from the tissue.

### DETAILED DESCRIPTION OF THE INVENTION

Autofluorescence endoscope systems to date demonstrate high sensitivity for the detection of cancerous or precancerous lesions. These areas are indicated by a reduction in the level of tissue autofluorescence. Visual detection of such regions is straightforward but often results in false positive readings since there are benign conditions which can cause the same effect. A method which results in a high number of false positive readings is described as one with low specificity. To improve the specificity of autofluorescence endoscopy additional information beyond a visual assessment of the reduction in fluorescence intensity can be taken. Spectral information, resulting from the dispersion or filtering of the intrinsic fluorescence and/or white light reflected from the tissue has been shown to be effective at diagnosing cancerous tissue. Similarly, information available from measurements of light scattering in the tissue can be used to classify tissue types and measure the concentration of important tissue components such as hemoglobin. This information has been correlated to the presence or absence of cancerous lesions. In the past such information has been available from fiberoptic spectral probes passed through the biopsy channel of an endoscope and brought into direct contact with the tissue under video observation.

There are numerous advantages of a non-contact spectroscopic probe used in conjunction with the autofluorescence endoscope. If the probe is built into the endoscope it is always available. If the probe does not contact the tissue it cannot damage the tissue surface, raise a layer of blood and thus cause a false positive reading of reduced fluorescence (which is readily absorbed by blood). If the area being examined spectroscopically can be indicated visually on the endoscope imaging display then the area can be readily positioned on the tissue by adjusting the direction of the distal tip. This disclosure described such a non-contact spectroscopic probe system. The design is such that it can be used in existing scopes by fitting it into a standard biopsy channel. The optical components required to be within the endoscope itself are small and passive and thus can be fit into new endoscope designs with minimal effort. All active light sources and detection systems are external to the endoscope. These may either be housed separately from the endoscope light source or built into it for a completely self-contained system.

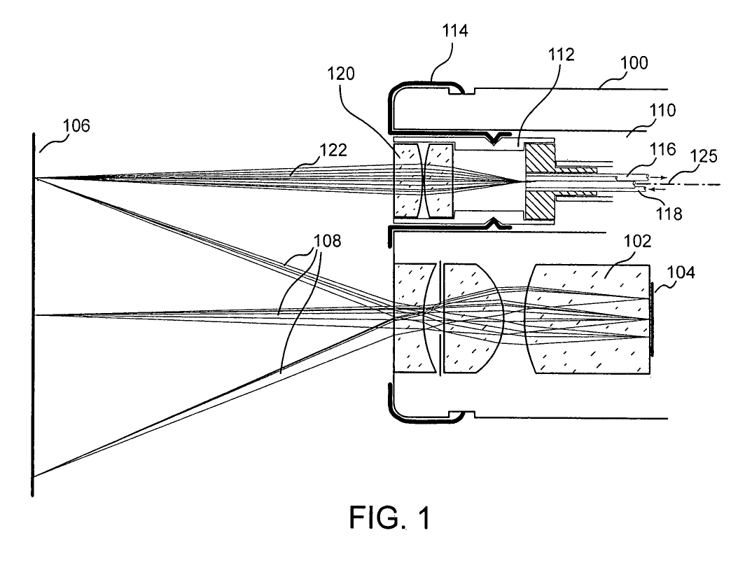

FIG. 1 shows a preferred embodiment of the non-contact probe's passive optical components as they are contained within an autofluorescence endoscope's distal tip, 100. In this schematic the optical components are contained within a 2.8 mm diameter biopsy probe channel of a video autofluorescence endoscope with an overall diameter at the distal tip of about 10 mm. The endoscope lens system 102 and video detector 104 provide a real-time image of the tissue surface 106. The optical ray bundles 108 indicate that the endoscope images the tissue surface over a wide field angle. The biopsy channel 110 contains the distal end of the non-contact probe's optical system 112 which is held in a fixed position by a holder such as the retaining clip 114. A set of matched detents in the retaining clip and the optical probe allow the probe to be inserted into the biopsy channel during a procedure, snapped into a fixed position and removed when it is necessary to use the channel for a normal biopsy.

The collection fibers 116 can be bonded together in a fixed array pattern with the delivery fibers 118. The polished ends of the collection fibers and delivery fibers are imaged onto the tissue by the probe's lens system 120 as indicated by the ray bundle 122. The collection and delivery fibers are nominally NA 0.22 fused silica fibers that can be used with an f/2 lens system 120 to efficiently couple them to the tissue. A single collection fiber and delivery fiber can be used for simple fluorescence and color measurements. For effective scattering measurements three collection fibers with a diameters of from 100 to 200 micrometers can be used with three delivery fibers of to 100 micrometer diameter. Smaller collection fibers generally do not collect sufficient light for many applications. Larger collection fibers can be too stiff to be built into the flexible distal tip of the endoscope. The delivery fibers are preferably coupled to laser sources and can work efficiently at diameters of 50 micrometers, for example. For coupling the illumination fibers to filtered thermal sources such as tungsten halogen bulbs or arc lamps, their diameters can be at least 100 micrometers.

The probe imaging lens set 120 preferably has a planar surface on the end facing the tissue so that liquid films have the least effect on focusing distance. The diameter of the lens set can be as small as 1 mm or as large as 2 mm with 1.5 mm being preferred for most applications. Smaller lenses are favored for incorporating the optical system permanently into an endoscope while the 2 mm size collects light more efficiently and still fits into a standard biopsy channel.

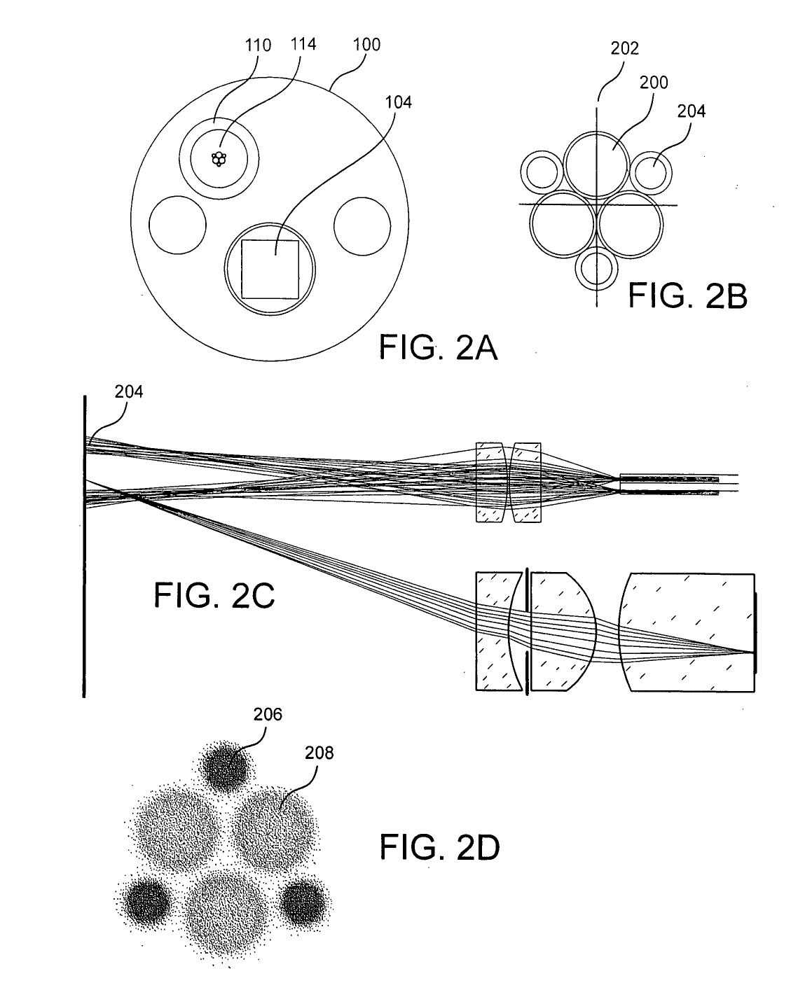

FIG. 2A shows an embodiment of the system in which a non-contact probe is positioned in the biopsy channel of an endoscope 110. The illumination and delivery fibers 114 are positioned on the axis of and parallel to the biopsy channel. Note that the biopsy channel axis 125 is typically off axis both horizontally and vertically from the endoscopes imaging system 104.

FIG. 2B shows a preferred arrangement of the collection and delivery fibers in a bundle which is as small as possible to keep them close to the optical axis of the imaging lens set. The three spots of the delivery fiber can also be easily distinguished in the visible field of the endoscope and serve as a marker for the position and the scale size of the area from which light is being collected for spectroscopy. In this triangular arrangement there are two collection fibers close to each distribution fiber and one at a greater distance. The two different distances can be important for the measurement of scattering in the tissue. This design has three-fold symmetry which works well with three-source, RGB imaging methods.

FIG. 2C shows how the delivery fibers are imaged onto the tissue in a three-fold spot pattern 204. This spot pattern is imaged by the endoscope detection system as shown in FIG. 2D. FIG. 2D also shows the effective collection areas for the three collection fibers on the tissue surface. Note that the three spots due to the delivery fibers 206 are distinct from the three spots from which the collection fibers collect light 208. This means that, when the probe is focused on the tissue, the collection fibers do not see reflected light from the delivery fibers but only light which has scattered through the tissue. This scattering measurement is preferably performed with the endoscope's other illumination sources, such as the broadband or white light imaging sources, turned off during tissue scattering measurements.

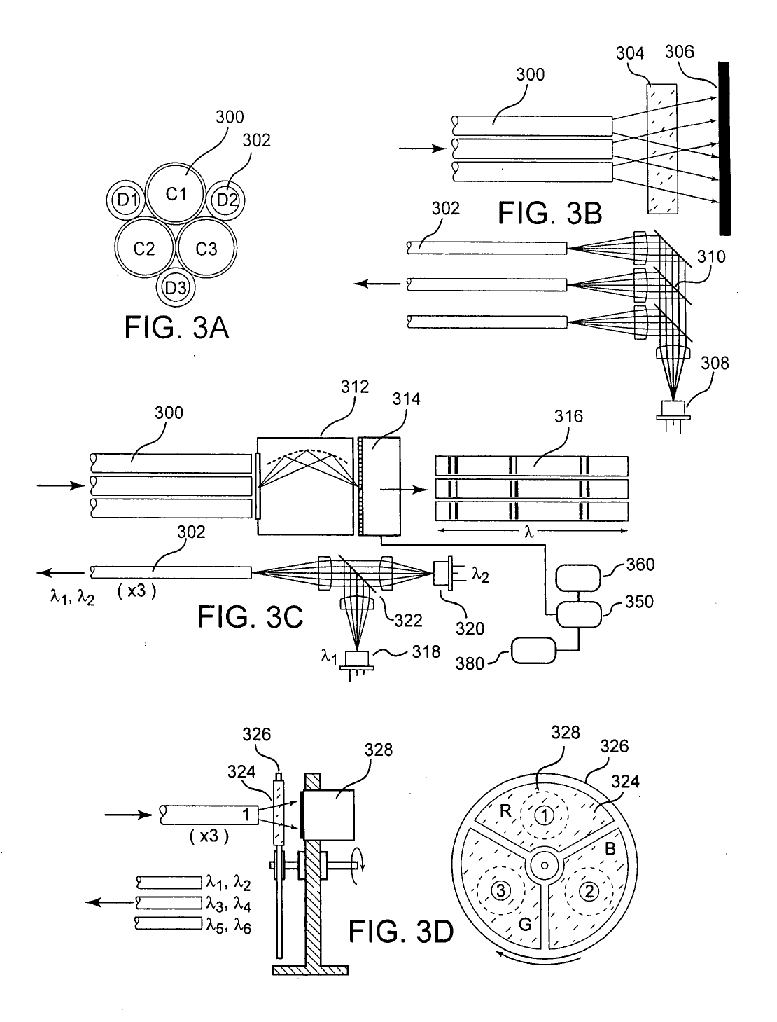

FIG. 3A repeats the preferred arrangement of the collection fibers 300 and the delivery fibers 302 with notations indicating that there can be, for some tissue measurements, important geometric relationships between specific delivery fibers and specific collection fibers. FIG. 3B shows a system for a simple spectroscopic detection method suitable for measuring absolute tissue fluorescence. The light from the three collection fibers 300 is combined and filtered with long-pass optical filter 304 to block the endoscope's fluorescence excitation light from reaching the single photodetector 306. Note that this simple collection system can be coupled to the same optical system in the endoscope as the more complex detection systems to be described hereinafter. The choice of which detection system to be used can depend on the tissue type to be diagnosed. In this simple detection system the delivery fibers 302 are only used to mark the position of the collection area on the tissue and in a preferred embodiment, three are coupled to the same illumination source such as a diode laser 308 using a lens and beamsplitter design 310.

FIG. 3C shows the preferred detection and illumination system based on an imaging spectrometer 312 and two-dimensional array detector 314 which yields three separate spectra 316 on the array detector, one for each collection fiber. The delivery fibers in this preferred system couple two or more wavelengths into a single fiber preferably using diode or solid-state lasers 318 and 320. In a preferred system, two wavelengths can be well separated on any single fiber allowing a dichroic beamsplitter 322 to be used for efficient coupling and mixing. In the preferred embodiment there are as many as six different laser lines coupled into the delivery fibers.

FIG. 3D shows an alternative detection system based on discrete photodetectors and filters which may have advantages for some tissue types in terms of cost or overall detection time. In this embodiment, the light from a single collection fiber is passed through an optical filter 322 contained in a rotating wheel 324 to a single photodetector 328. There are three such photodetectors, each matched to one of the three collection fibers. FIG. 3E shows an axial view of the rotating filter wheel which nominally carries red, green and blue filters. Each collection fiber thus sequentially detects red, green and blue colors which may be discrete as coupled into the delivery fibers. The specific detection sequences and preferred wavelengths are illustrated hereinafter. The systems described herein use a computer or data processor 350, a display 360 and a controller 380. The processor 350 can include a memory and a plurality of stored programs and databases to perform the various processes described herein. The display 360 can be used to display spectra and images of tissue as well as quantitative data derived from processing operations including the tissue characteristics described herein. A controller 380, either integrated with the computer, or constructed as a separate system can be used to control system operations including light sources, rotating filters and detectors.

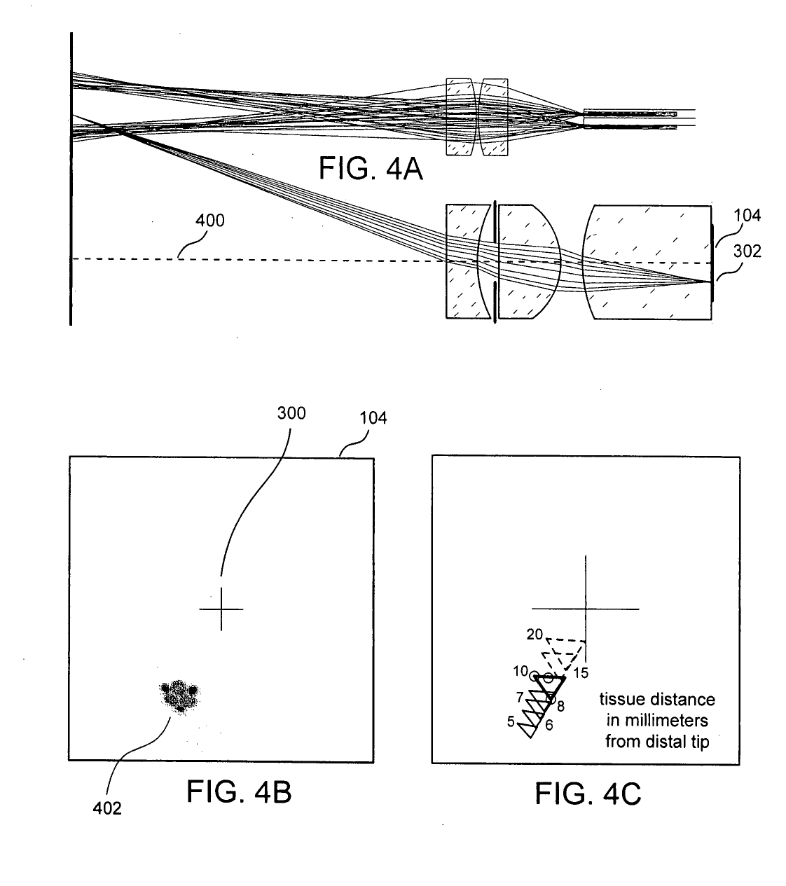

FIGS. 4A-4C show how the image of the three spot preferred marking or illumination pattern on tissue can be imaged on the endoscope's display system. The focus of the three spots is not critical but the scattering measurements will be optimal when the distal tip is one focal distance from the tissue. This preferred focus distance is set as 10 mm in these ray-tracing diagrams but may vary depending on the tissue type and location in the body. FIG. 4A shows that, in this endoscope system, the optical axis 400 of the endoscope imaging system is centered on the endoscope array detector 104 at position 402. FIG. 4B is a simulated display image with the optical axis marked by the cross at position 400 and the collection area of the tissue marked by the three illuminated spots at position 402. Since the optical probe is offset from the optical axis of the endoscope in both the vertical and horizontal planes, the image of the collection area is imaged down and to the left of the center of the display. The lateral position of the three spot marker varies with the distance of the endoscope's distal tip from the tissue as is shown by the ray-traced calculation in FIG. 4C. The shifting position of the three spot marker on the display allows the non-contact probe to be precisely focused onto the tissue. At the correct focal distance the three spot marker can be positioned around a fiducial mark on the endoscope display. The apparent motion as a function of focal distance that an image pattern recognition computer program looking for the particular colors of the three spot marker can determine the actual distance of the probe tip from the tissue and calculate the scale size of the delivery and collection spots, even if the probe is not precisely at the correct focal distance. Knowing the distance of the delivery spots from the collection spots is an important capability for the calibration of the tissue scattering measurements.

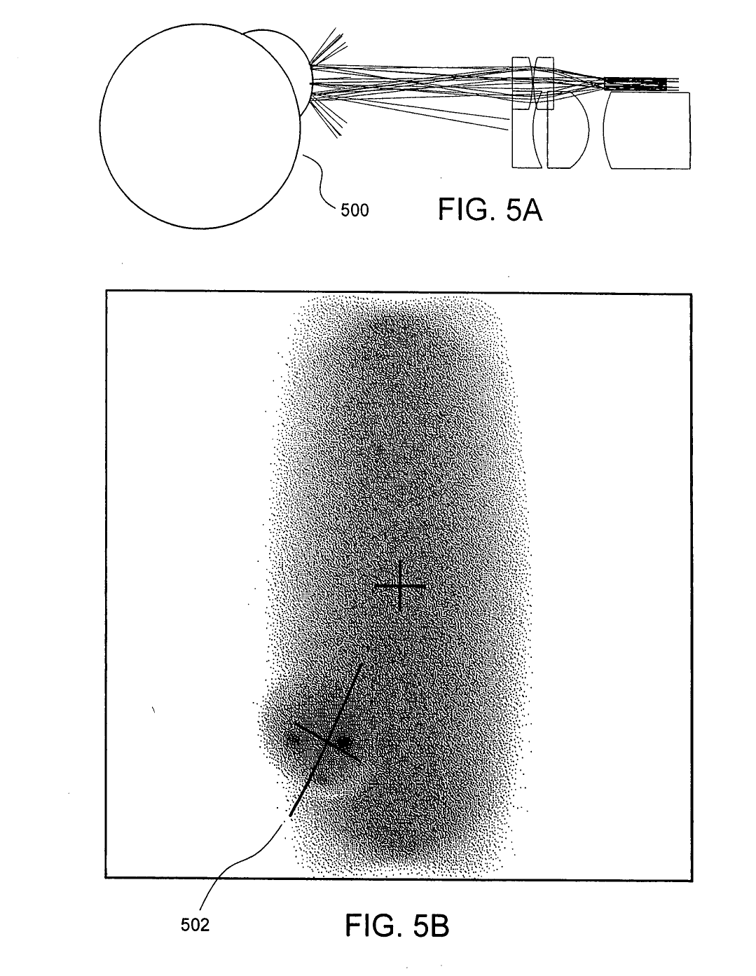

FIGS. 5A-5B show a simulated image (inverted for clarity) of a cylindrical scattering object 500 (FIG. 5A) with a spherical bump on its surface as it is seen on the endoscope display as imaged by the endoscope optics shown in FIG. 1. This example uses a distance of 8 mm for the distance of the distal tip of the endoscope to the tissue. As predicted by the calculations shown in FIG. 4 the spots are low and to the left of the optimal position marked by a fiducial 502 on the display screen of FIG. 5B. The line of positions where the spots can be seen by the endoscope is shown by the line extending from near the edge of the field towards the center of the field. This fiducial 502 can have additional marks showing the allowed range of tip/tissue distances for the scattering measurements where focus is important. Outside of this marked range the collection fibers collect directly reflected light from the delivery fibers which can be useful for other types of diagnostics.

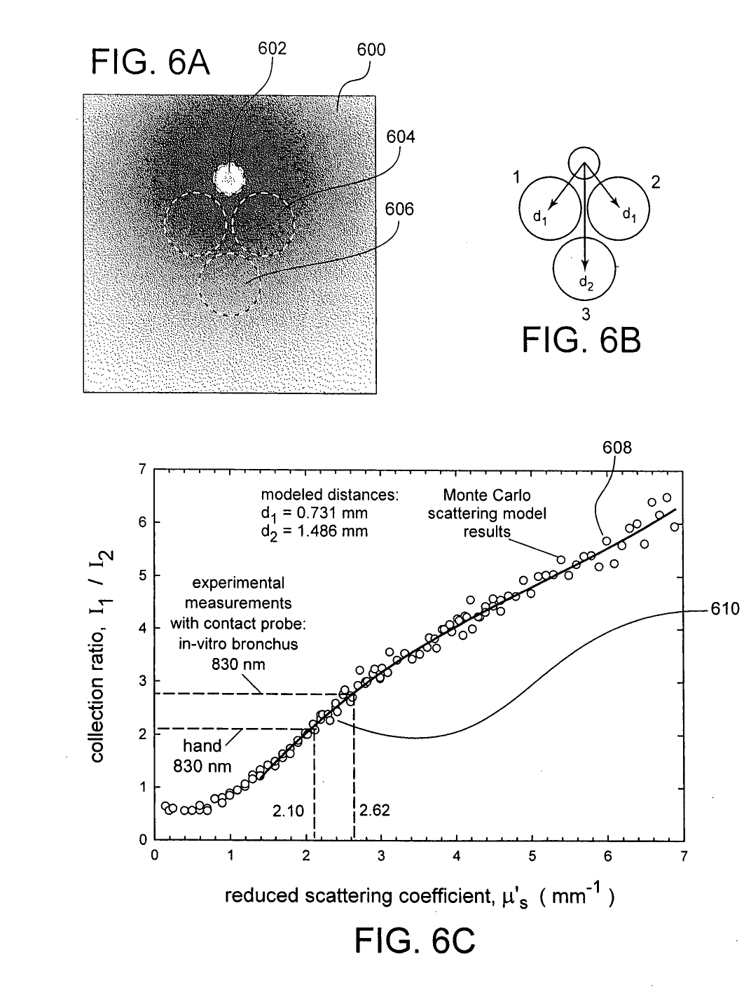

FIGS. 6A-6C show the results of tissue scattering at a single wavelength and how measurements of light from the delivery fibers coupled into the collection fibers through tissue scattering can be used to determine the characteristic scattering parameter of the tissue at that wavelength. Measurements of scattering characteristics at other wavelengths, which may be made simultaneously, can be used to measure the concentration of hemoglobin in the tissue. FIG. 6A shows how light entering the tissue at a delivery spot 602 exits the tissue surface at a distance due to scattering within the tissue. In this simulation the size of the calculated image of the tissue 600 is 2 mm on a side. The dashed circle 604 shows the collection area of a collection fiber lying next to that particular delivery fiber. The dashed circle 606 shows the collection area of a collection fiber lying opposite the delivery fiber in the preferred bundle arrangement. At the particular scattering parameter used in this example, the collection fibers close to the delivery fiber collect more photons than the collection fiber at a distance. This is not always the case. If the characteristic scattering length in the tissue is large, the photons may not be able to turn around fast enough to exit near their entrance point and will show up as a ring of light around the entrance point. FIG. 6B shows the two distances, d1 and d2, which characterize the centers of the collection fiber images from the center of the delivery fiber image. Note that these distances depend on focal distance and the distance of the distal tip of the endoscope from the tissue.

FIG. 6C shows a quantitative calculation of the light collected by the optical system of the non-contact probe given light launched into the delivery fiber of the probe assuming NA 0.22 fibers for both the delivery and collection probes. Since absolute measurements are difficult to make, it is preferable to measure ratios of collected signals. In FIG. 6C the ratio plotted is that of the signal received by the fiber at the greater distance d2 to the average of the signals received by the two fibers at the shorter distance d1. In this example, the reduced scattering coefficient was varied and two probe measurements were included. The mean free path, mfp, of the photons can also be varied with a fixed average scattering angle, θ, of 30 degrees. the effective mean free path, mfp′, as provided by scattering theory is given by mfp/(1−cos(θ)). The effective mean free path characterizes the distance a scattering photon has to travel to turn around in the tissue and thus exit.

The results shown in FIG. 6C as dots 608 on the graph are reasonably well fit by a straight line 610 in the scattering region of interest for tissue. The measurement system can thus be easily calibrated in practice using physical scattering standards as well as this type of representation. These results assume, in this graph, that there is no absorption in the tissue. In actual tissue there is absorption so that the signal from the more distant collection fiber will be smaller than the signal from the closer fiber. Absorption, in other words, will be characterized by a smaller collection ratio. Hemoglobin is the important absorber for diagnostic purposes. By measuring the tissue at two close wavelengths, one of which is absorbed by hemoglobin and one of which is not, the concentration of the hemoglobin can be determined. In practice, a series of measurements of scattering across the visible spectrum and near UV spectrum can be made and analyzed as a group.

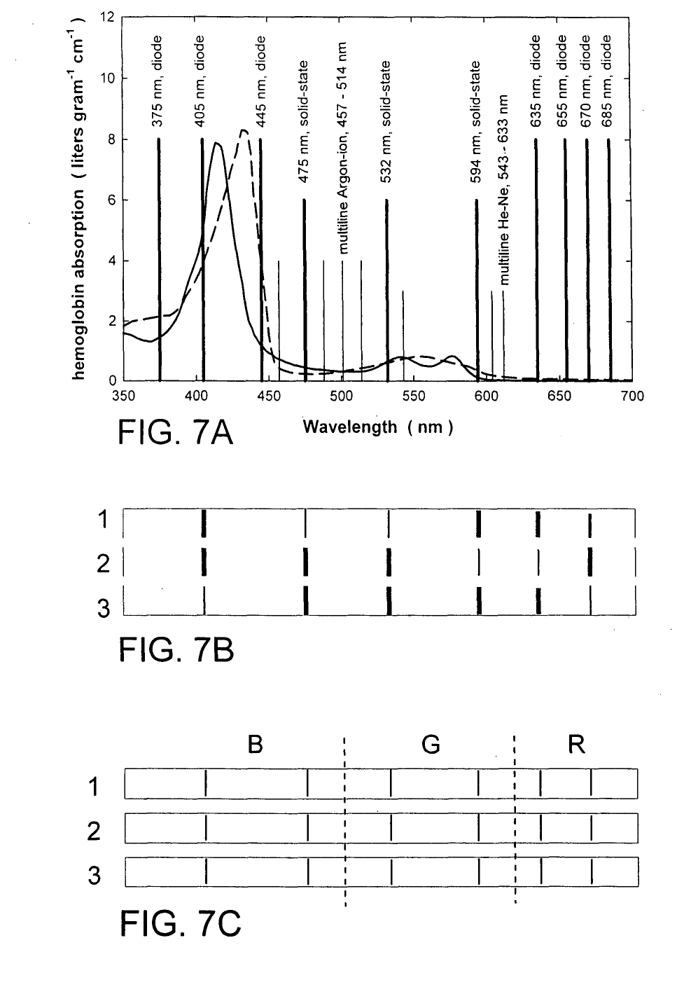

FIG. 7A shows a graph of hemoglobin absorption overlaid with important laser lines which are available from standard laser sources suitable for preferred embodiments of the present invention. Diode lasers have a long operating life and are preferred. Diode lasers are available at the red, violet and near UV regions of the spectrum. Diode-pumped solid state lasers are preferred for some wavelengths, particularly for green and yellow wavelengths. In the blue region suitable laser lines might be 405 nm where hemoglobin absorption is large and 475 nm where the absorption is relatively low. In the mid-visible region, 532 nm is highly visible and absorbed to some extent by hemoglobin and can be used with the yellow line at 594 nm where hemoglobin absorption is low At red wavelengths there is very little hemoglobin absorption and laser sources emitting at 635 nm and 670 nm can be used.

All of these wavelengths can be applied to the tissue simultaneously and their scattering measured simultaneously at both scatter distances using the imaging spectrometer system shown in FIG. 3C. There are a total of 18 wavelength/distance combinations with this system. FIG. 7B shows what the array detector coupled to the spectrometer can see in one example. The individual fibers at the entrance slit are imaged separately with this type of spectrometer. Heavy lines represent the signal expected from collection fibers closest to the delivery fiber in question. Lighter lines indicate the signal from collection fibers farther from the delivery fiber, both because of the added distance and because of possible absorption. The imaging spectrometer detection system can also measure the full spectrum of fluorescence from the tissue and if a cooled-CCD array is used, for example, the detection can be sensitive to a few photons. These CCD cameras, however, typically require about 1 second to read a full array.

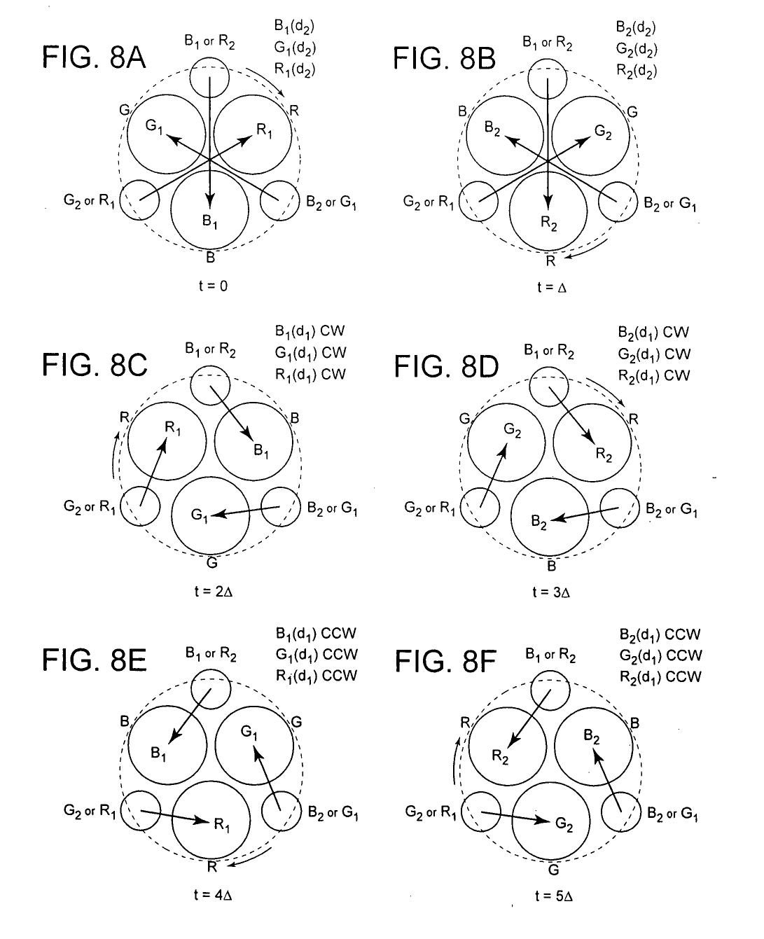

FIG. 7C shows that the measurement of all of these laser lines and distance combinations is more complex with discrete photodetectors. The measurements cannot be made simultaneously or without shifting the filters from one fiber/detector combination to the next. The detectors however, if photomultipliers are used, can be very sensitive. Their outputs can be integrated over a light pulse period and the resulting signal digitized directly and quickly for calculations. FIGS. 8A-8F show how the various combinations of colors and scattering distances can be measured with 6 separate light pulse periods resulting from two rotations of the colored filter wheel.

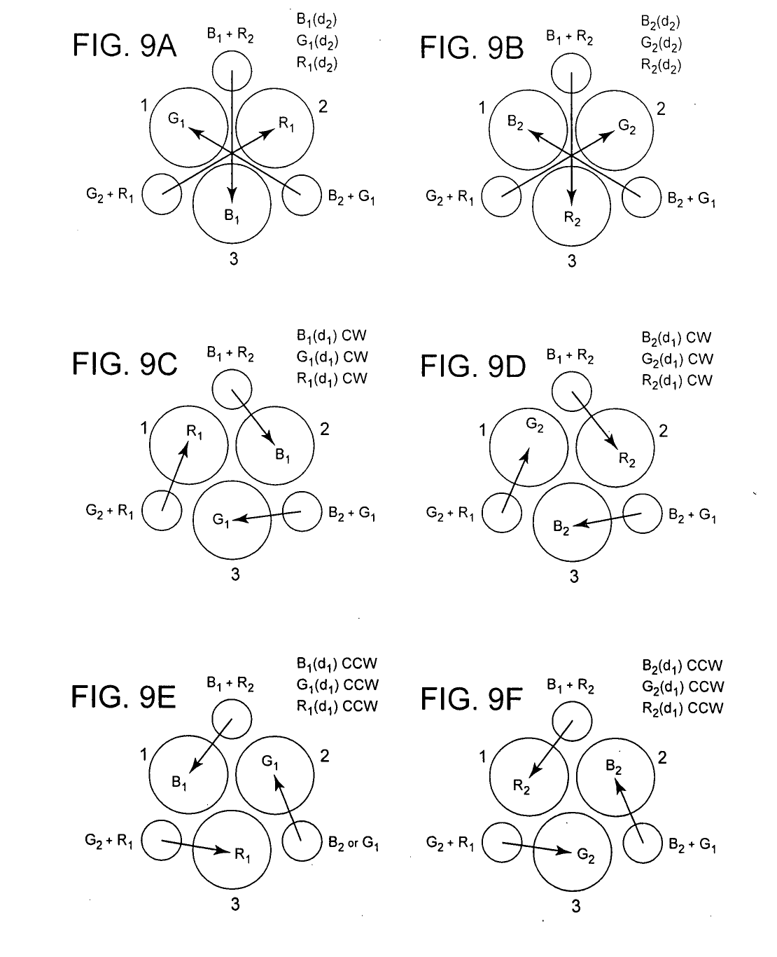

FIGS. 9A-9F show how a preferred embodiment of the detection system, using the imaging spectrometer, can detect all 18 of the wavelength/distance combinations resulting in the detector image shown in FIG. 7B. Note that with laser sources all of the returning photons end up in only a few pixels of the imaging detector, resulting in a high level of signal relative to readout noise.

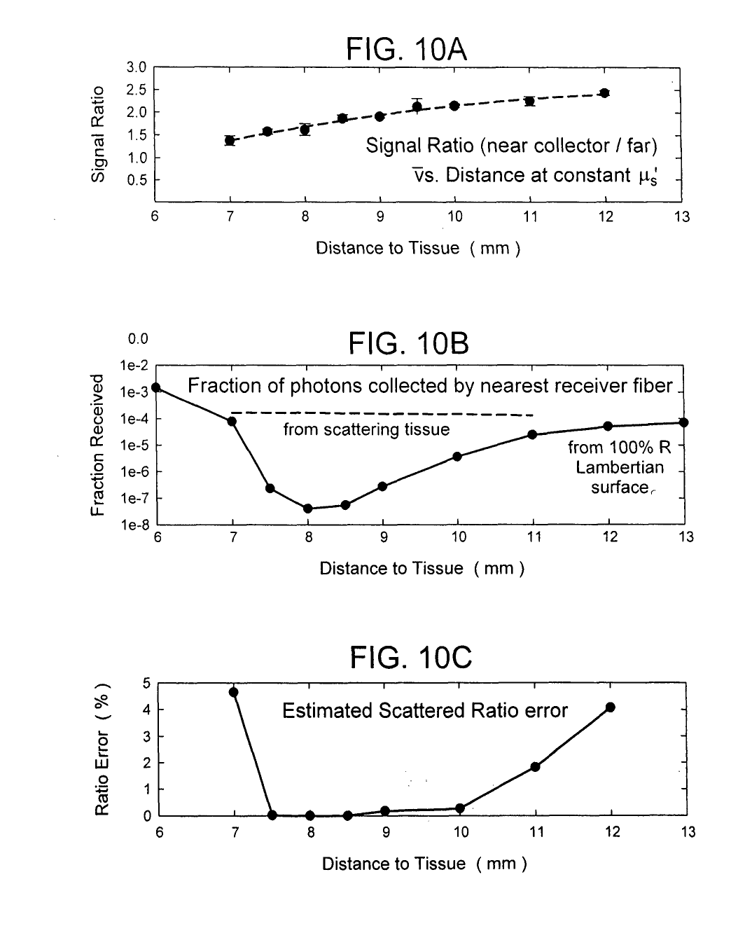

FIG. 10A shows the results of a tissue scattering model that can determine the expected variation in the scattering signal ratio (near collector/far collector), for a constant tissue mₛ′, as the probe is placed at varying distances from the tissue. The preferable position is at the desired focal distance (10 mm in this model), but a good measurement of the ratio is possible over a range of several millimeters. At slightly larger than the preferred tip-tissue distances, the effective separation of the light entrance and exit points increases so the far collector signal drops and the ratio of near/far signals increases. At tip-tissue distances much larger than the optimum, the entrance and exit spots begin to defocus and overlap. In this case there can be a direct back reflection of the entering light from the surface of the tissue into the collection fibers. Since this light has not passed through the tissue the result can introduce an error which will initially show up as an increase in the signal from the nearest collection fiber. FIG. 10B shows a calculation of this overlap error as a fraction of the near collection fiber signal as the tip-tissue distance in the model is varied over a large range. In FIG. 10B, the tissue surface is considered to be a 100% reflecting Lambertian diffusing surface. At the preferred focus distance, the error signal is over four orders of magnitude below the desired signal due to light scattering through the tissue. Real tissue is unlikely to scatter more than 10% of the incident photons in this way. With this assumption, the expected error in the measured scattering signal ratio can be estimated as shown in FIG. 10C. This estimate suggests a working range of +/−1.5 mm to keep the ratio error below about 1% for the through-tissue scattering measurements. FIG. 4C indicates that this working distance range can be easily seen in the video image of the endoscope so that the endoscope can be held at the proper distance.

While the invention has been described in connection with specific methods and apparatus, those skilled in the art will recognize other equivalents to the specific embodiments herein. It is to be understood that the description is by way of example and not as a limitation to the scope of the invention and these equivalents are intended to be encompassed by the claims set forth below.

1. A light scattering spectroscopic endoscope system comprising:
an optical probe for an endoscope having at least three illumination optical fibers and at least three collection optical fibers;
a lens assembly that couples light between a distal end of the collection optical fibers and tissue positioned at a distance from the distal end of the collection optical fibers;
an illumination light source optically coupled to proximal ends of the illumination fibers;
a detector system that senses a spectrum from light returned by the collection optical fibers; and
a data processor in communication with the detector system and storing instructions to process the spectrum for determining a tissue characteristic.

2. The system of claim 1 further comprising a holder that positions a distal end of the probe in a fixed position relative to a distal end of the endoscope.

3. The system of claim 2 wherein the holder further holds the distal end of the probe at a fixed angular orientation relative to an axis of the endoscope.

4. The system of claim 1 further comprising an endoscope having a channel for receiving the optical probe, the endoscope including an imaging device that generates image data such that a distance between the distal end of the probe and the tissue can be determined.

5. The system of claim 1 wherein the tissue characteristic is bulk scattering.

6. The system of claim 1 wherein the tissue characteristic is bulk absorption.

7. The system of claim 1 wherein the tissue characteristic is endogenous fluorescence.

8. The system of claim 1 wherein the tissue characteristic is apparent color.

9. The system of claim 1 wherein the returned light comprises reflected or fluorescent light in response to an endoscope illumination source.

10. The system of claim 1 wherein the illumination light source comprises a plurality of light sources.

11. The system of claim 1 wherein the plurality of sources coupled to the illumination fibers have a narrow spectral characteristic such that the sources can be identified with the detector system.

12. The system of claim 10 wherein the plurality of light sources are lasers.

13. The system of claim 1 wherein the detector system is a set of optical filters and discrete photodetectors.

14. The system of claim 12 wherein the optical filters rotate sequentially between collection fibers and their discrete photodetectors.

15. The system of claim 10 wherein the light sources are temporally modulated.

16. The system of claim 1 wherein the detector system is an imaging spectrometer.

17. The system of claim 1 wherein the system comprises an autofluorescence endoscope.

18. The system of claim 17 wherein the endoscope has a distal imaging device.

19. The system of claim 1 further comprising a system controller that controls one or more light sources and one or more detectors.

20. The system of claim 1 further comprising a first light source at a first wavelength and a second light source at a second wavelength.

21. The system of claim 1 further comprising an optical filter having red, green and blue components.

22. The system of claim 1 wherein the detector provides a spectrum from each collection fiber.

23. The system of claim 1 further comprising providing at least three light sources, one source coupled to a single optical fiber.

24. The system of claim 1 further comprising a fiducial marking system.

25. The system of claim 1 further comprising a pattern recognition program.

26. The system of claim 1 wherein three illumination fibers form a three spot pattern.

27. The system of claim 1 wherein each illumination fiber is paired with a collection fiber for correlated color measurement.

28. The system of claim 1 further comprising a distal lens system for a distally mounted imaging device including an aperture.

29. The system of claim 2 wherein the holder is at a distal end of the probe.

30. A method for spectroscopic measurement comprising:
providing an endoscopic device having a plurality of illumination optical fibers and a plurality of collection optical fibers;
coupling light to proximal ends of the illumination optical fibers;
using a lens assembly at a distal end of the endoscope to couple light from the illumination optical fibers onto a tissue surface positioned at a distance from the lens assembly;
collecting light from the tissue surface with the lens assembly and the collection optical fibers;
detecting light from each of the collection optical fibers with a detector system to provide a detected spectrum; and
processing the spectrum to determine a tissue characteristic.

31. The method of claim 30 further comprising determining a distance of the lens assembly from the tissue surface.

32. The method of claim 30 wherein the step of providing an endoscope device comprises providing an endoscope with a working channel and a probe for insertion in the working channel, the probe including the lens assembly, the illumination fibers and the collection fibers.

33. The method of claim 30 further comprising using the illumination fibers to form a light pattern on the tissue and using the image pattern to locate a distal end of the endoscope at a distance from the tissue.

34. The method of claim 33 further comprising using a fiducial marker with the image pattern to position the endoscope relative to the tissue.

35. The method of claim 32 further comprising attaching the probe to the endoscope with a holder.

36. The method of claim 30 further comprising using an endoscope imaging detector to measure a distance of the endoscope to a tissue surface

37. The method of claim 30 further comprising using pairs of illumination and collection fibers to emit and collect separate colors.

38. The method of claim 30 further comprising using the endoscope at a distance of not more than 1.5 mm more than a selected distance or 1.5 mm less then the selected distance.

39. The method of claim 30 further comprising using a plurality of diode lasers at different wavelengths to illuminate the tissue.

40. The method of claim 30 further comprising forming separate spectra from each collection fiber.
